(19) United States
(12) Patent Application Publication
(10) Pub. No.: US 2007/0161866 A1
(43) Pub. Date: Jul. 12, 2007

(54) Deep tissue blade for use with retractor systems

(75) Inventors: James M. Fowler JR., Houston, TX (US); Marc D. Gelnett, Houston, TX (US); Kevin G. Bowen, Houston, TX (US)

Correspondence Address: VINSON & ELKINS L.L.P., 1001 FANNIN STREET, 2300 FIRST CITY TOWER, HOUSTON, TX, 77002-6760, US

(73) Assignee: Lone Star Medical Products, Inc., Stafford, TX (US)

(21) Appl. No.: 11/329,909

(22) Filed: Jan. 11, 2006

(51) Int. Cl. A61B 1/32

(52) U.S. Cl. 600/233

(57) ABSTRACT

The present invention provides a deep tissue blade for use with a retractor system useful in a wide variety of surgical procedures. The deep tissue blade of the present invention also provides several unique structures that enhance its ability to retract and hold large tissue masses.

### BACKGROUND OF THE INVENTION

1. Field of the Invention

The present invention relates to surgical retractors and surgical retractor stays or blades. More particularly, the present invention relates to an annular retractor frame adapted for receiving and securing an end portion of an elastic member of a surgical retractor stay or blade. The present invention further relates to a bridge connecting two points or areas of the annular retractor frame which is adapted for receiving and securing an end portion of an elastic member of a surgical retractor stay or blade.

2. Description of Related Art

During the course of many surgical procedures, tissue surrounding an incision or wound is separated and retracted by means of retractors or stay sutures, which are generally held by trained assistants. Most retractors are one piece metallic implements that retract a wound in a non-yielding manner. Many retractors retract the wound in a manner such that manipulation and movement by the surgeon as well as movement caused by contracting muscles or tissues of the patient can result in bruising or tearing of the tissue. Once the tissue is separated and retracted, further stabilizing, retracting, or delivering of exposed tissues or organs, requires the placement of additional retractors or stay sutures. Nevertheless, the use of these sutures and attached devices can clutter the surgical field for the surgeon and can be difficult to maintain in their desired positions due to the variations in the skills of the operating staff, especially in cases where the surgery takes an extended period of time.

Several retractor systems have been developed that require less continual maintenance by the physician and staff. Many of these embody devices designed for a designated area of the body on which surgery is to be performed without causing undue visual or physical obstructions.

More recently, elastic surgical retractor systems have come into commercial use that include elastic stays, each having an elongated elastic member that is typically a hollow length of elastic tubing. The elastic tubing provides proximal and distal end portions. The distal end portion typically carries an elongated hook constructed of wire which is placed in the distal end of the bore of the hollow tubing. In many instances, a shrink wrap is placed over the hook and tubing to hold the proximal end of the wire hook firmly in position within the bore of the tubing at the distal end. The embedded portion of the wire hook member is usually recurved or folded. This folded proximal portion of the wire hook can expand the tubing slightly, forming a vertically extended portion that defines a handle.

Various patents have issued for elastic stay retractor systems. A surgical retractor array system is disclosed in U.S. Pat. No. 4,434,791, issued to W. Dale Darnell on Mar. 6, 1984. This surgical retractor system comprises an array of standardized, interchangeable, annular retractor frame sections of various shapes of which the end portions are configured to permit the interchangeable, hinged connection of the various shaped frames in forming generally annular retractor units adaptable to conform to fit the surface contours of various patients upon which a surgical operation is to be performed. This retractor frame is designed to accept yielding rubber or like elastic stays.

Other patents have issued that relate to elastic type retractor stays and related retractor frames and systems. For example, U.S. Pat. No. 4,274,398, issued to Frank B. Scott, Jr., on Jun. 23, 1981, which is hereby incorporated by reference, discloses a surgical retractor which includes an annular frame conformed to fit the surface contour of the portion of the body to be operated on. At least one stay includes an elastic member and a tissue holding hook. The frame has a plurality of notches spaced about its periphery. The elastic portion of the stay is in the form of a length of hollow elastic tubing adapted to be inserted into one of the notches of the frame and held in place by friction to retract the tissue. The hook is a single, curved wire member. It has a folded proximal end that fits the hollow bore of the elastic tube.

U.S. Pat. No.: RE 32,021, issued to Frank B. Scott, Jr., on Nov. 5, 1985, which is hereby incorporated by reference, discloses a surgical retractor which includes a frame conformed to fit the surface contour of the portion of the body to be operated on and at least one stay which includes an elastic member and tissue holding means. The frame, furthermore, has a plurality of notches spaced about its periphery and the elastic member of the stay is adapted to be inserted into one of the notches and held in place by friction to retract the tissue.

In addition, United States Patent Application Pub No.: US2005/0171405A1, published on Aug. 4, 2005, which is hereby incorporated by reference, provides a surgical retractor system having annular frame and at least one support member attached to the annular frame in a notch based attachment system, wherein at least a portion of the support member extends over at least a portion of the inner area of the frame.

Despite these disclosures, a need remains for a retractor system that provides a maximized area for stable attachment of elastic stays and is capable of securely lifting and holding large tissue segments without causing undue injury to such tissue.

### SUMMARY OF THE INVENTION

The deep tissue blade of the present disclosure provides a blade for use with a retractor frame that includes an elastic tether attachment area having at least one attachment structure. The blade further includes a curved section that is connected to the attachment area. In certain embodiments the blade also includes a fulcrum structure on the distal surface of the attachment area. In still other embodiments, the blade can include a guide that directs an elastic tether toward the proximal end of the attachment area. In certain of these embodiments the fulcrum structure and the guide can be the same structure.

In certain embodiments, the deep tissue blade the curved section includes at least one reflexed edge or beaded edge to increase its strength. While in still others, the distal end of the curved section is blunted, flared or includes a traction enhancing feature selected from the group consisting of: ribs, spikes and hatching etches.

In certain embodiments the curved section can be shaped as an open triangle, an open box, or an open polygon. In some embodiments, the distal end of the curved section is comprised of spikes or fingers.

### DESCRIPTION OF THE FIGURES

FIG. 1 shows a retractor frame and several elastic stays.

FIG. 2 shows a retractor frame with a hinged region.

FIG. 3 shows an elastic stay.

FIG. 4 depicts the use of a retractor frame and elastic stays during a surgical procedure.

FIG. 5 shows one embodiment of a retractor frame with a bridge member.

FIG. 6 shows one embodiment of a bridge member.

FIG. 7 shows one embodiment of a deep tissue blade.

FIG. 8 shows a retractor frame being used with one embodiment of a deep tissue blade.

FIG. 9 shows an alternate embodiment of a deep tissue blade.

### DESCRIPTION OF THE INVENTION

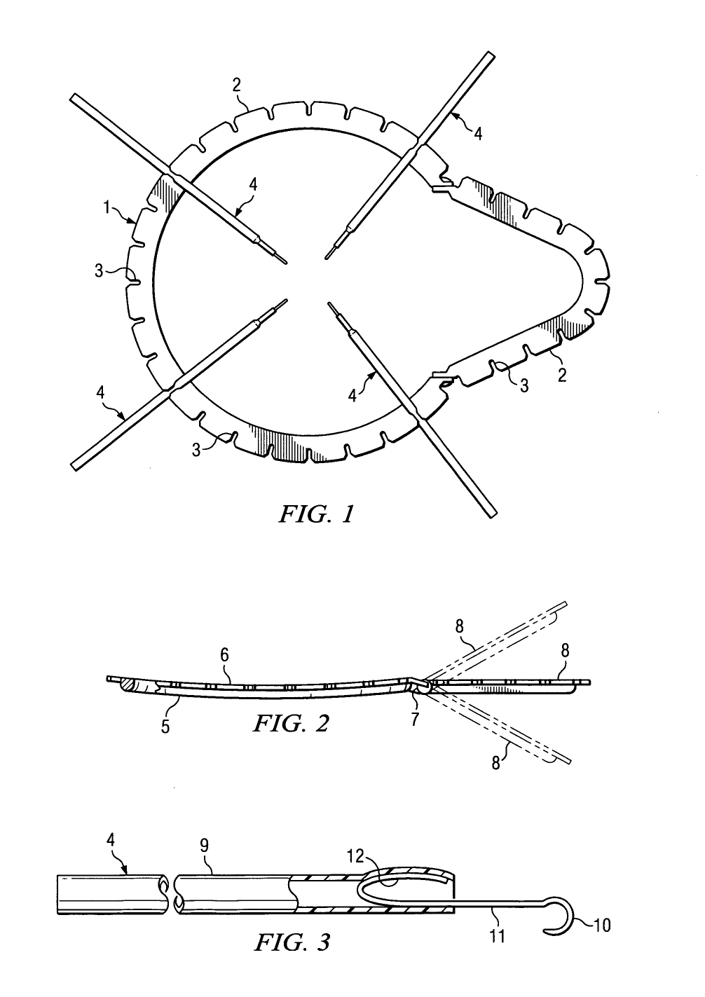

The present invention includes a retractor having a generally annular frame 1. One embodiment is depicted in FIGS. 1 and 2. In certain embodiments, the outer edge of the retractor 2 includes at least one, and preferably a plurality, of notches 3. The notches 3 are operable as connection points between the retractor frame 1 and at least one stay 4. The notches 3, in some embodiments, can be modified to hold or retain sutures.

The annular frame 1 further includes a proximal side 5 and a distal side 6. The retractor frame 1 of the present invention can be formed of any suitable material. In certain embodiments, the frame is composed of a non-pliable material. In some embodiments, the retractor frame 1 is reusable and composed of a material that can be sterilized and re-sterilized, such as stainless steel, titanium, aluminum, or other suitable metals. In alternate embodiments, the retractor frame 1 is disposable and composed of a material such as a plastic material that is sufficiently strong to support the use of multiple stays that are connected thereto without altering the shape of the frame 1. These embodiments can be pre-sterilized and packaged for delivery to the surgeon.

In certain embodiments the frame 1 may be shaped such that the distal side 5 of the frame 1 conforms to fit the surface contours of a specific portion of the body to be operated upon (as shown in FIGS. 2 and 4). In certain of these embodiments, the annular frame includes a hinged region 7 and an outwardly extending flanged portion 8 which can be utilized to further contour the annular frame to the region of the body to be operated on. The extending flanged portion 8 of the frame 1 can be maintained in any intermediate position by friction within the hinged region 7 between it and frame 1, or by other suitable mechanical means well-known in the art such as a thumb-screw or wing-nut. Furthermore, the flanged portion 8 can be any shape, including, but not limited to, circular, triangular or non-symmetrical configurations.

Alternate embodiments of the present invention include an annular frame 1 composed of suitable semi-pliable or malleable material(s). Suitable semi-pliable materials include materials that are capable of being molded into a particular shape while at the same time possessing enough resistance to shape changes to allow proper tension to be exerted on a retractor by an elastic stay or blade. Such materials include, but are not limited to, certain plastics and/or malleable metals such as nitinol. In these embodiments, the shape of the retractor may be modified to correspond to the contour of the bodily region to be operated on.

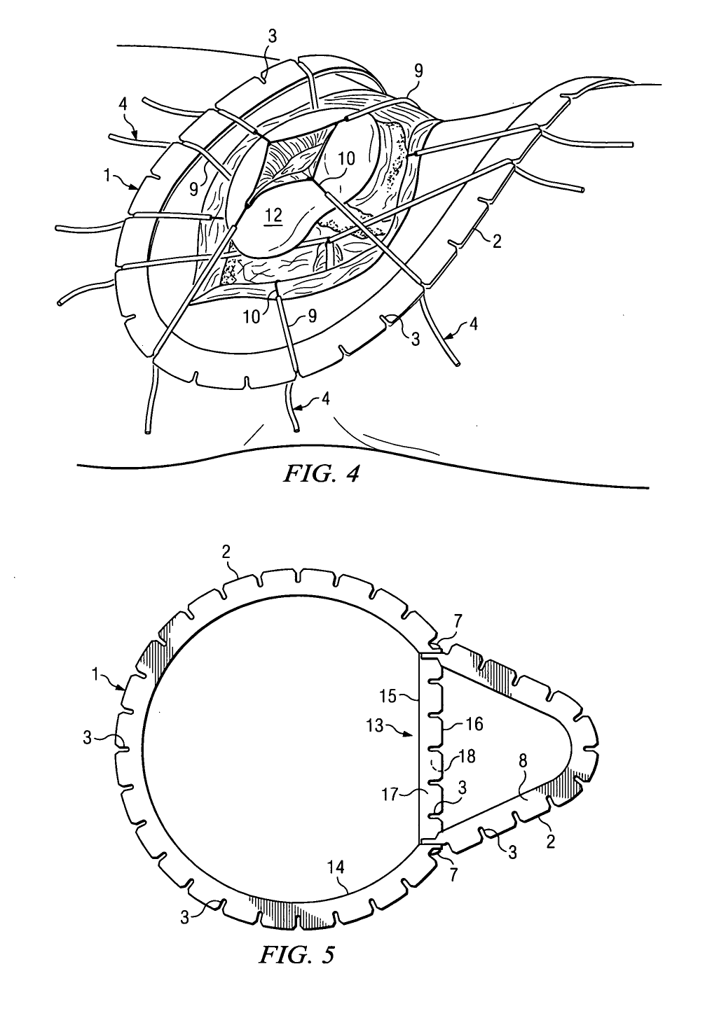

In certain embodiments, the frame 1 (including the outwardly extending flange portion 8 if present) may include a plurality of notches 3 about the outer edge. In some of these embodiments, the width and depth of each notch 3 is such that elastic portion 9 of a stay 4 (as shown in FIG. 3) may be inserted therein and held in place by friction without damaging the elastic portion 9.

In certain embodiments of the present invention, each stay 4 includes an elongated, flexible elastic member 9 that is connected to a tissue-holding device 10. In some embodiments, the elastic member 9 comprises a section of silicon rubber tubing or solid silicone cylinder. In still other embodiments, the stay includes a suture or other suitable line in place of the silicon rubber tubing. The tissue-holding device may be a hook 10 as shown in FIG. 3. In certain of these embodiments, the hook 10 includes a shank portion 11 and a recurved handle portion 12. The recurved handle portion 12 can be inserted into the lumen of the elastic member 9 for those embodiments utilizing silicon rubber tubing. The shape of handle portion 12 can be designed to provide a convenient means by which a surgeon can manipulate hook 10.

The operation of the retractor of the present invention is depicted in FIG. 4 which illustrates the use of retractor 1 to expose the bladder 12. After making an initial incision, a surgeon would use a pair of stays 4 to open the wound. The surgeon can control the tension applied to the edges of the wound by the tension or stretching of the elastic member 9 of the stays 4 when secured in the notches 3. As the surgeon proceeds through deeper layers of tissue, the wound is pulled further open by the tension of elastic members 9, and if the direction or force of the tension that is necessary needs to be changed, the surgeon can quickly do so simply by removing and reinserting the tissue-holding device, such as a hook 10 and/or by moving elastic member 9 to a different notch 3.

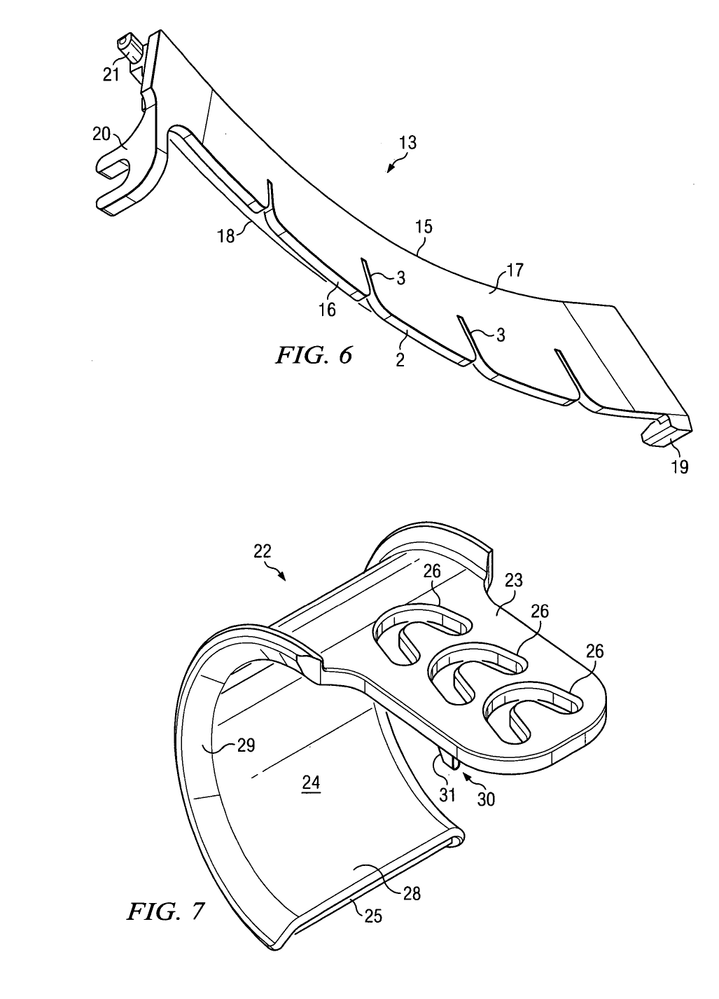

In certain embodiments, the retractor of the present invention also includes a bridge member 13 that can be used to cross or bridge at least a portion of the inner opening of the annular retractor frame (as shown in FIG. 5). The bridge member 13 can be made of the same types of materials as the retractor frame 1. The bridge member 13 can also be sterilizable and therefore reusable or can be provided in a disposable format. Furthermore, the bridge member 13 can be provided in a multitude of shapes and sizes. One embodiment of such a bridge member 13 is depicted in FIGS. 5 and 6 having an inner edge 15 and an outer edge 16, as well as a proximal surface 17 and a distal surface 18. In alternative embodiments, the bridge member can include a hinged section between its two ends allowing the structure to partially or completely fold in upon itself.

In certain embodiments, the bridge member removably connects between two points on the inner edge 14 of the retractor frame 1. In some embodiments, the bridge member 13 has an inner edge 15 and an outer edge 16 with respect to proximity to the incision location. The inner edge 15 and/or the outer edge 16, in some of these embodiments, includes at least one, and preferably a plurality, of notches 3. In certain embodiments the retractor frame 1 and bridge member 13 include a plurality of notches sufficient to provide a surgeon with incremental attachment points for the elastic member of a stay(s) or sutures.

In those embodiments of the retractor frame 1 that include a hinged region 7 and an extending flanged portion 8, the bridge member can connect across the inner area of the retractor frame from one hinged region 7 to another as depicted in FIG. 5. The bridge member 13 can connect to the retractor frame 1 through any suitable mechanism. For example, the bridge member 13 can be designed to have a mating portion on each end that would allow the bridge member to snap into a variety of locations within the retractor frame 1. In such embodiments, the retractor frame 1 would also include structures, such as slots, preconfigured for accepting the mating portion of the bridge member 13.

In other embodiments such as the one depicted in FIG. 6, the bridge member can be secured to the hinged region through a thumb-screw or wing-nut type mechanism. In such embodiments, the bridge member can further include a seating structure 19 located on one end for tethering or stabilizing the bridge member 13 to one side of the hinged region and a securing structure 20 on the other end for securing the bridge member to the retractor frame 1. The securing structure 20 can be of any configuration that will provide sufficient stability in the attachment of the bridge member 13 to the retractor frame 1. In certain embodiments, bridge member can have an additional seating structure 21 on either or both ends.

In alternate embodiments, the bridge member 13 can be a permanent feature of the retractor frame 1. In certain of these embodiments, the bridge member 13 is non-moveable. For example, the bridge member 13 can be permanently affixed to the retractor frame 1 or can be cast as a unitary portion of the retractor frame 1. In some of these embodiments, the bridge member 1 is permanently attached (either affixed thereto or cast as an integral feature) to the retractor frame 1 on one side. In such embodiments, the free end of the bridge member 13 can be reversibly attached to the frame through any suitable method.

The present invention further provides a deep tissue or Deaver-like blade for use with the retractor frame 1 and/or the bridge member 13. FIG. 7 depicts one embodiment of the deep tissue blade 22. The deep tissue blade includes a retractor attachment area 23 and a curved section 24. In certain embodiments, the deep tissue blade can also include an extension section from the proximal end of the curved section 24. The attachment area 23 is used to tether or attach the blade to the retractor frame 1 by attaching to an elastic tether through an attachment structure. In certain embodiments, the attachment structure includes at least one curved slot 26 for attachment of an elastic tether 27 as shown in FIG. 8. In certain of these embodiments, the attachment area 23 includes three curved slots 26 that may be utilized individually or in combination. One of ordinary skill in the art will readily understand a variety of configurations could be used to attach the elastic tether to the attachment area and that such structures are encompassed by the present disclosure.

In certain embodiments, the attachment area also includes guides 30 for the elastic tether(s) 27 (see FIG. 7). These guides 30 can be of any shape or size sufficient to prevent the elastic tether from rolling under the guide 30 during use. In certain embodiments, the guide 30 includes a beveled edge 31 extending proximally toward the end of the attachment area and a flat section that can be used as a fulcrum when the guide 30 is used in such a fashion as to make contact with the retractor frame 1 providing a downward force on the deep tissue blade 22 to pull the tissue laterally away from the surgical opening. In embodiments containing a guide 30, the elastic tether 27 can be looped onto the interior of one of the curved slots 26 and run inside the guide 30 to attach to the deep tissue blade 22. The guides 30 will keep the elastic tether 27 underneath the attachment area 23 from its point of attachment 26 until the elastic tether 27 is proximal of the guides 30. This distance paired with the force of the tether will create a moment arm that directs the distal end 25 of the curve 24 downward and lateral to help it hold the tissue. The elastic tether 27 can then be used to determine the tension the deep tissue blade 22 puts on the tissue during use by adjusting the length of the elastic tether 27 between the deep tissue blade 22 and the attachment of the elastic tether 27 to the retractor frame 1 or a notch 3 in the retractor frame 1 (as shown in FIG. 8). In still other embodiments, the attachment area 23 includes a fulcrum structure (without the guiding aspect with respect to the elastic tether 27) located on the distal side or edge of the attachment area 23 to provide a downward force on the deep tissue blade 22 to pull the tissue laterally from the surgical opening. A guide 30 or a fulcrum structure can be positioned anywhere on the distal surface or outer edge of the attachment area 23 to provide a variety of guiding functionalities or fulcrum forces. In certain embodiments the fulcrum structure is distal to the actual point of attachment for the elastic tether 27. Furthermore, the fulcrum structure can be in a number of shapes or sizes, that would be readily ascertainable by one of ordinary skill in the art. For example, the fulcrum structure can be in the form of a rail extending across the attachment area or a series of one or more pyramids on the distal surface of the attachment area. In some embodiments the guide 30 can act as the fulcrum structure. In still other embodiments the fulcrum structure is an integral part of the guide 30.

The distal end 25 of the curved section 24 can have any shape. In certain embodiments, the distal end 25 is blunted or rounded, while in still others it can be flared (either away from or toward the attachment area 23). Furthermore, the interior surface 28 of the curved section 24 can have a variety of traction enhancing features. For example, the interior surface can also include at least one rib, spike or hatching to enhance its ability to secure tissue during use.

The curved section 24, in certain embodiments, also includes a reflexed or beaded edge 29. In some of these embodiments, the beaded edge 29 prevents the deep tissue blade from cutting into the tissue and/or reduces potential distortions of the curved section during use. One of ordinary skill in the art will readily recognize that multiple shapes and features, which are encompassed by the present disclosure, would provide these same functions.

The deep tissue blade 22 can be of any size suitable for use with a given retractor frame 1. In certain embodiments, the internal opening between the distal end 25 of the curved section 24 and the distal surface of the attachment area 23 (or extension area in certain embodiments) can range from 20 mm to 25 cm, while in preferred embodiments it can range from 3 cm to 10 cm. In certain of these embodiments, the width of the deep tissue blade 22 can also range from 2 cm to 8 cm. In some of these embodiments, the width of the curved section 24 can be greater than the width of the attachment area 23.

The length of the curved section 24 can vary depending upon the deep tissue blades intended application and the distance between the internal opening between the distal end 25 of the curved section 24 and the distal surface of the attachment area 23. In certain embodiments, the length of the curved section can range from 3 cm to 25 cm, while in preferred embodiments it can range from 6 cm to 12 cm.

The thickness of the deep tissue blade can vary depending upon its material composition and should be sufficient to minimize or eliminate shape distortions during use. In certain embodiments, the thickness of the deep tissue blade can range from 0.05 to 2 inches for embodiments composed of Noryl® plastic, aluminum, steel. In certain embodiments, the deep tissue blade 22 is composed of 0.1 inch thick disposable Noryl®.

In certain alternate embodiments, the shape of the curved section 24 of deep tissue blade 22 can vary from an arch having a range of 90 to 200 degrees to a triangular apex with varying angles and side lengths. In still other embodiments, the shape of the curved section can be determined by a series of flat or relatively flat sections joined at angles to one another (open polygon). For example the curved section in some embodiments can resemble an open-ended box.

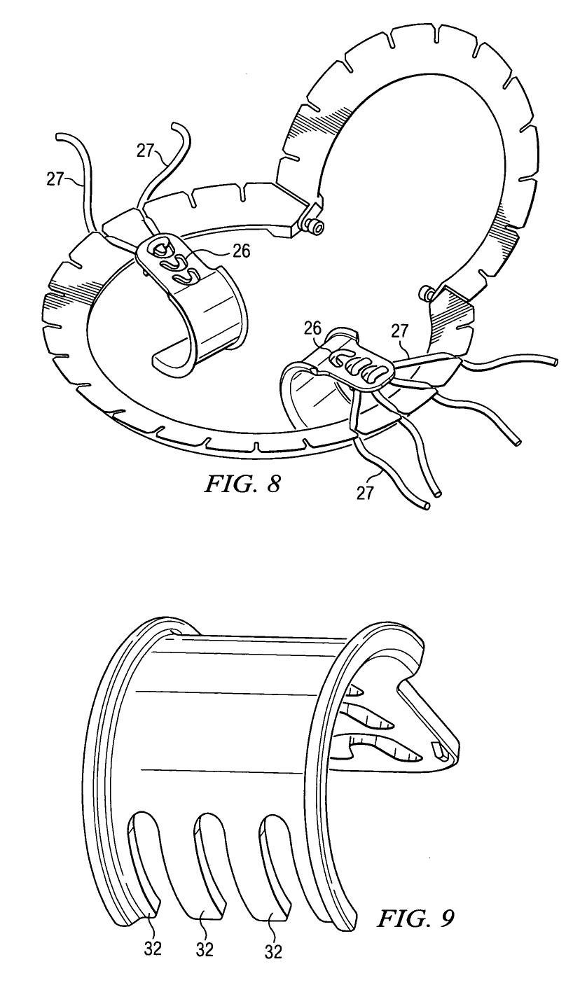

In certain embodiments, the curved section 20 can be separated into fingers 32 giving the deep tissue blade 22 a claw-like appearance such as shown in FIG. 9. In still other embodiments, the curved section can be divided into a series of spikes or spindles decreasing the actual contact area between the deep tissue blade 22 and the tissue while maintaining a wide section of tissue retraction during use. In certain of these embodiments, the deep tissue blade 22 also includes raised ridges on the finger 32 to increase resistance to deformation.

In still other embodiments, the deep tissue blade 22 can include a spacer section between the attachment area 23 and the curved section 24. The spacer section can be of any suitable length for use with a given sized retractor frame 1. In certain embodiments, the spacer section can range from greater than 0 cm to 5 cm.

From the foregoing, it can be seen that the retractor of the present invention increases greatly the efficiency and effectiveness of the surgeon. At least one member of the surgical team is eliminated, thereby reducing the expense to the patient. Moreover, by eliminating the need for an assistant directed by the surgeon, the surgeon is able to proceed more quickly, which results in further cost savings. Also, substantial benefits to the physical wellbeing of the patient are achieved by the use of the retractor of the present invention. The precise control that the surgeon has over the placement of and tension applied to the retracting devices minimize trauma during the course of the operation.

Further modifications and alternative embodiments of the retractor of this invention will be apparent to those skilled in the art in view of this description. Accordingly, this description is to be construed as illustrative only and is for the purpose of teaching those skilled in the art the manner of carrying out the invention. Various changes that may be made in the shape, size and arrangement of parts are encompassed by the correct disclosure.

What is claimed:

1. A deep tissue blade for use with a retractor frame comprising:
an elastic tether attachment area having a proximal surface, a distal surface, a proximal end and at least one attachment structure; and
a curved section having a proximal surface and distal surface that is connected to the attachment area.

2. The deep tissue blade of claim 1 further comprising a fulcrum structure on the distal surface of the attachment area.

3. The deep tissue blade of claim 1 further comprising a fulcrum structure extending distally from an outer edge of the distal side of the attachment area.

4. The deep tissue blade of claim 1 further comprising a guide that directs an elastic tether toward the proximal end of the attachments area.

5. The deep tissue blade of claim 4, wherein the guide further acts as a fulcrum structure.

6. The deep tissue blade of claim 1, wherein the curved section includes at least one reflexed edge or beaded edge.

7. The deep tissue blade of claim 1, wherein the distal end of the curved section is blunted.

8. The deep tissue blade of claim 1, wherein the distal end of the curved section is flared toward the attachment area.

9. The deep tissue blade of claim 1, wherein the distal end of the curved section is flared away from the attachment area.

10. The deep tissue blade of claim 1, wherein the distal surface of the distal end of the curved section further includes a traction enhancing feature selected from the group consisting of: ribs, spikes and hatching etches.

11. The deep tissue blade of claim 1, wherein the curved section has a shape selected from the group consisting of: an open triangle, an open box, or an open polygon.

12. The deep tissue blade of claim 1, wherein at least a portion of the distal end of the curved section is comprised of spikes or fingers.

13. A deep tissue blade for use with a retractor frame comprising:
an elastic tether attachment area having a proximal surface, a distal surface, a proximal end and at least one attachment structure for an elastic tether;
at least one elastic tether guide on the distal surface of the attachment area that directs the elastic tether when attached toward the proximal end of the attachment area; and
a curved section having a proximal surface, distal surface, and distal end that is connected to the attachment area.

14. The deep tissue blade of claim 13 further comprising a fulcrum structure on the distal surface of the attachment area.

15. The deep tissue blade of claim 14 wherein the fulcrum structure and the guide are the same structure.

16. The deep tissue blade of claim 13, wherein the curved section includes at least one reflexed edge or beaded edge.

17. The deep tissue blade of claim 13, wherein the distal end of the curved section is blunted.

18. The deep tissue blade of claim 13, wherein the distal end of the curved section is flared toward the attachment area.

19. The deep tissue blade of claim 13, wherein the distal end of the curved section is flared away from the attachment area.

20. The deep tissue blade of claim 13, wherein the distal surface of the distal end of the curved section further includes a traction enhancing feature selected from the group consisting of: ribs, spikes and hatching etches.

21. The deep tissue blade of claim 13, wherein the curved section has a shape selected from the group consisting of: an open triangle, an open box, or an open polygon.

22. The deep tissue blade of claim 13, wherein at least a portion of the distal end of the curved section is comprised of spikes or fingers.

23. A deep tissue blade for use with a retractor frame comprising:
an elastic tether attachment area having a proximal surface, a distal surface, a proximal end and at least one attachment structure for an elastic tether;
at least one elastic tether guide on the distal surface of the attachment area that directs the elastic tether when attached toward the proximal end of the attachment area;
a curved section having a proximal surface, distal surface, and distal end that is connected to the attachment area; and
a fulcrum structure on the distal side of the attachment area.

24. The deep tissue blade of claim 23 wherein the fulcrum structure and the guide are the same structure.

25. The deep tissue blade of claim 23, wherein the curved section includes at least one reflexed edge or beaded edge.

26. The deep tissue blade of claim 23, wherein the distal surface of the distal end of the curved section further includes a traction enhancing feature selected from the group consisting of: ribs, spikes and hatching etches.

27. The deep tissue blade of claim 23, wherein the curved section has a shape selected from the group consisting of: an open triangle, an open box, or an open polygon.

28. The deep tissue blade of claim 23, wherein at least a portion of the distal end of the curved section is comprised of spikes or fingers.
